(19) United States
(12) Patent Application Publication
(10) Pub. No.: US 2020/0093751 A1
(43) Pub. Date: Mar. 26, 2020

(54) CHEMOPROTECTIVE/CHEMOACTIVE NANODROPLETS AND METHODS OF USE THEREOF

(71) Applicant: Der-Yang TIEN, Pasadena, CA (US)

(72) Inventor: Der-Yang TIEN, Pasadena, CA (US)

(21) Appl. No.: 16/313,929

(22) PCT Filed: Jun. 27, 2017
(86) PCT No.: PCT/US17/39377

§ 371 Date: Dec. 28, 2018

Related U.S. Application Data
(60) Provisional application No. 62/355,388, filed on Jun. 28, 2016.

(51) Int. Cl. A61K 9/51; A61K 31/355; A61K 47/60; A61K 31/337; A61P 35/00; A61K 9/00

(52) U.S. Cl. CPC A61K 9/5146; A61K 31/355; A61K 47/60; A61K 9/0019; A61K 9/5123; A61K 31/337; A61P 35/00; A61K 9/5138

(57) ABSTRACT

Disclosed herein are nanodroplets containing (i) a tocotrienol, (ii) a tocopherol or tocotrienol covalently bonded to a polyalkylene glycol, (iii) a poloxamer, and (iv) a polyalkylene glycol. In certain aspects, the nanodroplets described herein have anti-cancer activity even in the absence of an anti-cancer agent (e.g., chemotherapeutic agents). Thus, the nanodroplets described herein can be used alone or in combination with one or more anti-cancer agents to treat cancer. The nanodroplets exhibit low toxicity, are biodegradable, and offer chemoprotective effects when administered alone or alongside traditional anti-cancer agents. Furthermore, the nanodroplets do not interfere with the efficacy of anti-cancer agents and result in a greater reduction of tumor volume when administered to subjects with cancer as compared to commercially-available products alone.

### CROSS REFERENCE TO RELATED APPLICATIONS

This application claims priority upon U.S. provisional application Ser. No. 62/355,388 filed Jun. 28, 2016. This application is hereby incorporated by reference in its entirety.

### BACKGROUND

Delivery of hydrophobic drugs to the appropriate tissues in the body has long been a challenge for medical researchers, who must maximize biocompatibility while minimizing toxicity. An ideal delivery vehicle would avoid premature release of its cargo, thereby delivering a larger dose of the drug to the effective site. Further, it is highly desirable to avoid affecting non-target tissue in order to maximize treatment of the target area as well as to avoid adverse systemic effects. This is of particular concern in cancer research, where many anti-cancer chemotherapeutic agents are hydrophobic and can have toxic side effects.

Further, a chemoprotective agent that reduces the side effects of a chemotherapeutic agent without affecting its therapeutic effect would have significant clinical benefits. A number of natural and synthetic compounds have been shown to be chemoprotective; for example, amifostine has some chemoprotective effects against cisplatin-related renal toxicity and neutropenia due to cisplatin-cyclophosphamide combination therapy. However, amifostine's side effects, including hypotension, nausea, and vomiting, plus the possibility that it quenches cisplatin's activity and/or lower cisplatin's efficacy, have limited its usage. Therefore, medical researchers have a continued interest in finding significantly improved chemoprotectors without similar risks of side effects.

What is needed is a delivery vehicle that results in slower drug release due to increased stability upon dilution during circulation, better tumor growth inhibition, fewer systemic side effects including lower hematological toxicity, better tumor targeting, and greater bioavailability. Additionally, a chemoprotective agent that does not diminish the chemotherapeutic effects of anti-cancer drugs is needed. Ideally the aforementioned delivery vehicle could carry chemoprotective agents directly to tumor sites.

### SUMMARY

Disclosed herein are nanodroplets containing (i) a tocotrienol, (ii) a tocopherol or tocotrienol covalently bonded to a polyalkylene glycol, (iii) a poloxamer, and (iv) a polyalkylene glycol. In certain aspects, the nanodroplets described herein have anti-cancer activity even in the absence of an anti-cancer agent (e.g., chemotherapeutic agents). Thus, the nanodroplets described herein can be used alone or in combination with one or more anti-cancer agents to treat cancer. The nanodroplets exhibit low toxicity, are biodegradable, and offer chemoprotective effects when administered alone or alongside traditional anti-cancer agents. Furthermore, the nanodroplets do not interfere with the efficacy of anti-cancer agents and result in a greater reduction of tumor volume when administered to subjects with cancer as compared to commercially-available products alone.

The advantages of the materials, methods, and devices described herein will be set forth in part in the description that follows, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below.

FIGS. 1A and 1B shows dynamic light scattering results evaluating particle size (A) without d-T3 and (B) with d-T3 in nanodroplet formulations such as those described herein. The Z-average diameter of nanodroplets without d-T3 is about 14 nm and for nanodroplets with d-T3 is about 30 nm.

FIG. 2 shows the effect of nanodroplet formulations on relative body weight for nude mice without tumors. Groups of mice (n=5) were injected through the tail vein twice per week with different dosages of d-T3 per injection. At the end of the trial, all groups of mice increased in body weight, indicating low toxicity of the nanodroplet formulations.

FIG. 3 shows tumor growth inhibition efficiency in a model of untreated mice, TAXOTERE® treated mice, and mice treated with nanodroplets containing d-T3 (Tonp) using an NCI-H460 human lung cancer model in nude mice. NCI-H460 cell suspensions were injected subcutaneously on the backs of mice to establish a tumor model. When tumor volume reached approximately 50 mm³, groups of tumor-bearing mice (n=5) were injected through the tail vein once per week for a total of two weeks with a dose of 15 mg/kg per animal for TAXOTERE® or 30 mg/kg per animal twice per week for three weeks for Tonp nanodroplets. On the 20^{th} day, the tumor growth inhibition of d-T3 (Tonp) treatment was 70%. Thus, treatment with d-T3 was at least as effective as TAXOTERE® treatment over the course of the study period.

FIG. 4 shows the effect of treatment on relative body weight in a model of untreated mice compared to mice treated with nanodroplets containing d-T3 (Tonp) using an NCI-H460 human lung cancer model in nude mice. When tumor volume reached approximately 50 mm³, groups of tumor-bearing mice (n=5) were injected through the tail vein twice per week for a total of three weeks with a dose of 30 mg/kg per animal. At the end of the trial, d-T3 treated mice did not have a significantly different body weight from control mice.

FIG. 5 shows the lack of toxicity of d-T3 nanodroplets versus untreated mice and mice treated with TAXOTERE® alone using an NCI-H460 human lung cancer model in nude mice. When tumor volume reached approximately 50 mm³, groups of tumor-bearing mice (n=5) were injected through the tail vein twice per week for a total of three weeks with a dose of 30 mg/kg per animal of Tonp or once per week with a dose of 15 mg/kg per animal of TAXOTERE®. By about two weeks post treatment, mice in the TAXOTERE® group had decreased in body weight, while the d-T3 (Tonp) treated mice did not have significantly different body weights from untreated controls.

FIG. 6 shows the chemoprotective effect of d-T3 nanodroplets in combination with TAXOTERE® treatment versus untreated controls or TAXOTERE® alone using an NCI-H460 human lung cancer model in nude mice. When tumor volume reached approximately 50 mm³, one group of tumor-bearing mice (n=5) were injected with TAXOTERE® through the tail vein once per week at a dose of 15 mg/kg per animal compared with another group of tumor-bearing mice (n=5), which were injected with TAXOTERE® and Tonp through the tail vein once per week at the dose of 15 mg/kg and 20 mg/kg, respectively, per animal. The Tonp was injected one day before TAXOTERE® starting just before the second injection of TAXOTERE®. After two injections of TAXOTERE®, the group without Tonp showed a severe body weight drop, and the treatment thus ended on the 13^{th} day of the study. However, one injection of Tonp inhibited the drop in body weight and created a chance for extended treatment. TAXOTERE® treatment resulted in a significant amount of weight loss; this was mediated by simultaneous treatment with d-T3 nanodroplets.

FIG. 7 shows tumor growth inhibition efficiency of d-T3 nanodroplets in combination with TAXOTERE® treatment versus untreated controls or TAXOTERE® alone in a NCI-H460 human lung cancer model in nude mice. NCI-H460 cell suspensions were injected subcutaneously on the backs of mice to establish the tumor model. When the tumor volume reached approximately 50 mm³, one group of tumor-bearing mice (n=5) were injected with TAXOTERE® through the tail vein once per week at a dose of 15 mg/kg per animal compared with another group of tumor-bearing mice (n=5), which were injected with TAXOTERE® and Tonp through the tail vein once per week at a dose of 15 mg/kg and 20 mg/kg, respectively, per animal. The Tonp was injected one day before TAXOTERE® starting just before the second injection of TAXOTERE®. After two injections of TAXOTERE®, the group without Tonp suffered a severe body weight drop and the treatment ended on the 13^{th} day with 71% tumor growth inhibition. For the group treated with TAXOTERE® and Tonp, on the 19^{th} day of the study, the tumor growth inhibition of the combined treatment was 76%, showing the synergistic effect of Tonp with TAXOTERE®. Tumor growth was inhibited approximately equally in both TAXOTERE® groups, showing that d-T3 use does not decrease the efficacy of TAXOTERE®.

FIG. 8A shows the disappearance of a tumor in a nude mouse treated with TAXOTERE® and protected with d-T3 nanodroplets (Tonp treatment). NCI-H460 cell suspensions were injected subcutaneously on the backs of mice to establish the tumor model. When tumor volume reached approximately 50 mm3, each mouse was injected with a dose of 15 mg/kg TAXOTERE® on day 1, 20 mg/kg of Tonp on day 6, and 15 mg/kg of TAXOTERE® on day 7. On the 11^{th} day of treatment, the tumor had disappeared and did not recur by the end of the trial. FIG. 8B shows the nude mouse whose tumor was cured by this treatment.

### DETAILED DESCRIPTION

Before the present materials, articles, and/or methods are disclosed and described, it is to be understood that the aspects described below are not limited to specific compounds, synthetic methods, or uses, as such may, of course, vary. It is also to be understood that the terminology used herein is for the purpose of describing particular aspects only and is not intended to be limiting.

In the specification and in the claims that follow, reference will be made to a number of terms that shall be defined to have the following meanings:

It must be noted that, as used in the specification and the appended claims, the singular forms “a,” “an,” and “the” include plural referents unless the context clearly dictates otherwise. Thus, for example, reference to “an anti-cancer agent” includes mixtures of two or more such anti-cancer agents, and the like.

“Optional” or “optionally” means that the subsequently described event or circumstance can or cannot occur, and that the description includes instances where the event or circumstance occurs and instances where it does not. For example, the compositions described herein may optionally contain one or more lyoprotectants, where the lyoprotectant may or may not be present.

As used herein, the term “about” is used to provide flexibility to a numerical range endpoint by providing that a given value may be “a little above” or “a little below” the endpoint without affecting the desired result. For example, the term “about” can be ±10% of a specified value (e.g., “about 20 wt %” includes 18 wt % to 22 wt %).

Throughout this specification, unless the context dictates otherwise, the word “comprise,” or variations such as “comprises” or “comprising,” will be understood to imply the inclusion of a stated integer or step or group of integers or steps but not the exclusion of any other integer or step or group of integers or steps. It is also contemplated that the term “comprises” and variations thereof can be replaced with other transitional phrases such as “consisting of” and “consisting essentially of.”

“Admixing” or “admixture” refers to a combination of two components together when there is no chemical reaction or physical interaction. The terms “admixing” and “admixture” can also include the chemical interaction or physical interaction among any of the components described herein upon mixing to produce the composition. The components can be admixed alone, in water, in another solvent, or in a combination of solvents.

The term “subject” as defined herein is any organism in need of cancer treatment and/or prevention. In one aspect, the subject is a mammal including, but not limited to, humans, domesticated animals (e.g., dogs, cats, horses, and the like), livestock (e.g., cows, pigs, and the like), experimental animals (e.g., mice), and wild animals.

The term “treat” as used herein is defined as maintaining or reducing the symptoms of a pre-existing condition. For example, the compositions described herein can be used to reduce the volume of tumor in a subject, reduce or prevent the rate of tumor growth, and the like.

The term “inhibit” as used herein is the ability of the compositions described herein to completely eliminate an activity or reduce the activity when compared to the same activity in the absence of the compound. For example, the compositions described herein can be used to inhibit the growth and/or spread of cancers in the body of a subject.

“Biodegradable” materials are capable of being decomposed by bacteria, fungi, or other organisms, or by enzymes in the body of a subject.

“Biocompatible” materials are materials that perform their desired functions without eliciting harmful or deleterious changes to the subject in which they are implanted or to which they are applied, either locally or systematically. In one aspect, the compositions disclosed herein are biocompatible.

As used herein, “toxicity” refers to harmful effects a substance has on an organism such as a human or mammal, or on cells within that organism. A compound or composition with high toxicity would be unsuitable for use as a medical treatment, while a compound or composition with low toxicity would be acceptable for use as a medical treatment. In one aspect, the compounds and compositions disclosed herein exhibit low toxicity.

References in the specification and concluding claims to parts by weight, of a particular element in a composition or article, denote the weight relationship between the element or component and any other elements or components in the composition or article for which a part by weight is expressed. Thus, in a compound containing 2 parts by weight of component X and 5 parts by weight of component Y, X and Y are present at a weight ratio of 2:5, and are present in such a ratio regardless of whether additional components are contained in the compound. A weight percent of a component, unless specifically stated to the contrary, is based on the total weight of the formulation or composition in which the component is included.

As used herein, a plurality of items, structural elements, compositional elements, and/or materials may be presented in a common list for convenience. However, these lists should be construed as though each member of the list is individually identified as a separate and unique member. Thus, no individual member of any such list should be construed as a de facto equivalent of any other member of the same list based solely on its presentation in a common group, without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range was explicitly recited. As an illustration, a numerical range of “about 1 to about 5” should be interpreted to include not only the explicitly recited values of about 1 to about 5, but also to include individual values and sub-ranges within the indicated range. Thus, included in this numerical range are individual values such as 2, 3, and 4, the sub ranges such as from 1-3, from 2-4, from 3-5, etc., as well as 1, 2, 3, 4, and 5 individually. The same principle applies to ranges reciting only one numerical value as a minimum or a maximum. Furthermore, such an interpretation should apply regardless of the breadth of the range or the characteristics being described.

Disclosed are materials and components that can be used for, can be used in conjunction with, can be used in preparation for, or are products of the disclosed compositions and methods. These and other materials are disclosed herein, and it is understood that when combinations, subsets, interactions, groups, etc., of these materials are disclosed, that while specific reference to each various individual and collective combination and permutation of these compounds may not be explicitly disclosed, each is specifically contemplated and described herein. For example, if a tocotrienol is disclosed and discussed and a number of different poloxamers are discussed, each and every combination of tocotrienol and poloxamer that is possible is specifically contemplated unless specifically indicated to the contrary. For example, if a class of molecules A, B, and C are disclosed, as well as a class of molecules D, E, and F, and an example combination of A+D is disclosed, then even if each is not individually recited, each is individually and collectively contemplated. Thus, in this example, each of the combinations A+E, A+F, B+D, B+E, B+F, C+D, C+E, and C+F is specifically contemplated and should be considered from disclosure of A, B, and C; D, E, and F; and the example combination of A+D. Likewise, any subset or combination of these is also specifically contemplated and disclosed. Thus, for example, the sub-group of A+E, B+F, and C+E is specifically contemplated and should be considered from the disclosure of A, B, and C; D, E, and F; and the example combination of A+D. This concept applies to all aspects of the disclosure including, but not limited to, steps in methods of making and using the disclosed compositions. Thus, if there are a variety of additional steps that can be performed with any specific embodiment or combination of embodiments of the disclosed methods, each such combination is specifically contemplated and should be considered disclosed.

Described herein are chemoprotective/chemoactive nanodroplets. The components used to produce the nanodroplets as well as methods of making and using the nanodroplets is provided below.

### Tocotrienol

One or more tocotrienols (component (a)) are present in the nanodroplets disclosed herein. In one aspect, the tocotrienol is alpha-tocotrienol, beta-tocotrienol, gamma-tocotrienol, delta-tocotrienol, or any combination thereof. In one aspect, the tocotrienol is a purified isomer (e.g., greater than 95%, greater than 99%, or 100% delta-tocotrienol). The D- and L-isomers of each tocotrienol (d-T3) can be used herein. In one aspect, the naturally-occurring D-isomers of tocotrienol (e.g., D-delta tocotrienol or D-gamma-tocotrienol) can be used herein. The structure of the naturally-occurring D-isomers of tocotrienol are provided in Table 1 below.

### Tocopherol or Tocotrienol Covalently Bonded to a Polyalkylene Glycol

The nanodroplets disclosed herein include a tocopherol or tocotrienol covalently bonded to a polyalkylene glycol (component (b)). Each component of component (b) and modes of bonding are further discussed below.

### Tocopherol or Tocotrienol

In one aspect, the tocopherol or tocotrienol of component (b) is a member of the vitamin E family of compounds. In another aspect, the tocopherol or tocotrienol is alpha-tocopherol, beta-tocopherol, gamma-tocopherol, delta-tocopherol, alpha-tocotrienol, beta-tocotrienol, gamma-tocotrienol, delta-tocotrienol, or a combination thereof. Structures of these compounds are provided in Table 1. The D- and L-isomers of each tocotrienol or tocopherol can be used herein with respect to component (b). Table 1 below provides the structures of the D-isomers of naturally-occurring tocopherols and tocotrienols useful herein.

TABLE 1
Tocopherol and Tocotrienol Structures

| | |
|---|---|
| alpha- tocopherol | 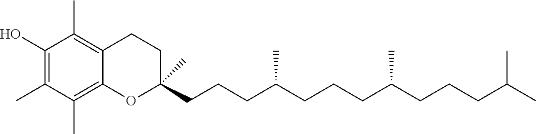 [SMILES: Cc1c(C)c2c(c(C)c1O)CC[C@@](C)(CCC[C@H](C)CCC[C@H](C)CCCC(C)C)O2] |
| beta- tocopherol | 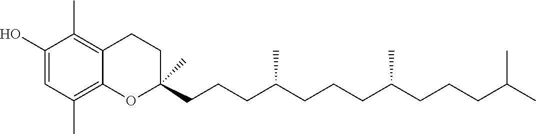 [SMILES: Cc1cc(O)c(C)c2c1O[C@](C)(CCC[C@H](C)CCC[C@H](C)CCCC(C)C)CC2] |
| gamma- tocopherol | 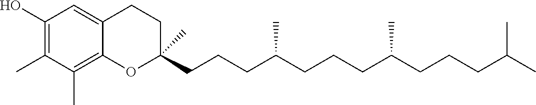 [SMILES: Cc1c(O)cc2c(c1C)O[C@](C)(CCC[C@H](C)CCC[C@H](C)CCCC(C)C)CC2] |
| delta- tocopherol | 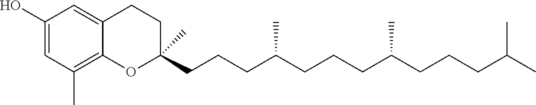 [SMILES: Cc1cc(O)cc2c1O[C@](C)(CCC[C@H](C)CCC[C@H](C)CCCC(C)C)CC2] |
| alpha- tocotrienol | 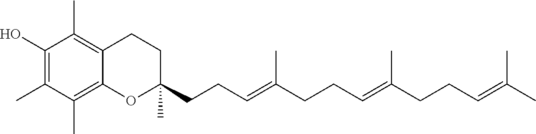 [SMILES: CC(C)=CCC/C(C)=C/CC/C(C)=C/CC[C@]1(C)CCc2c(C)c(O)c(C)c(C)c2O1] |
| beta- tocotrienol | 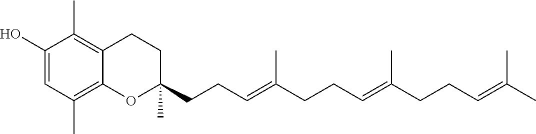 [SMILES: CC(C)=CCC/C(C)=C/CC/C(C)=C/CC[C@]1(C)CCc2c(C)c(O)cc(C)c2O1] |
| gamma- tocotrienol | 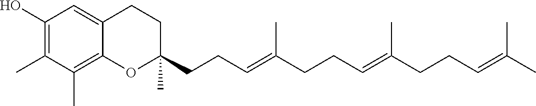 [SMILES: CC(C)=CCC/C(C)=C/CC/C(C)=C/CC[C@]1(C)CCc2cc(O)c(C)c(C)c2O1] |
| delta- tocotrienol | 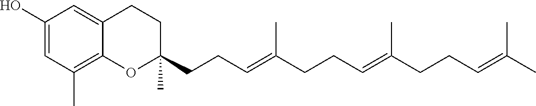 [SMILES: CC(C)=CCC/C(C)=C/CC/C(C)=C/CC[C@]1(C)CCc2cc(O)cc(C)c2O1] |

### Polyalkylene Glycol

In one aspect, the polyalkylene glycol covalently bonded to the tocopherol or tocotrienol can be a homopolymer of ethylene oxide, a homopolymer of propylene oxide, a block copolymer or reverse block copolymer of ethylene oxide and propylene oxide, or a random copolymer of ethylene oxide and propylene oxide.

In another aspect, the polyalkylene glycol has an average molecular weight of from 200 to 2,000 Daltons. In another aspect, the polyalkylene glycol has an average molecular weight of 200, 300, 400, 500, 600, 700, 800, 900, 1000, 1100, 1200, 1300, 1400, 1500, 1600, 1700, 1800, 1900, or 2000 Daltons, where any value can be an upper and/or lower endpoint of a range (e.g., 500 to 1500, 800 to 1200, etc.). In one aspect, the polyalkylene glycol is a homopolymer of ethylene glycol and has an average molecular weight of at or about 1,000 Daltons.

### Linkers

In some aspects, the tocopherol or tocotrienol is covalently bonded to the polyalkylene glycol via a linker molecule. In one aspect, the linker is a polyfunctional carboxylic acid or anhydride. In a further aspect, the polyfunctional carboxylic acid or anhydride has the general formula II, III, or IV, as seen in Table 2:

TABLE 2
General Formulas of Linker Molecules

| | |
|---|---|
| 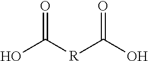 | II |
| 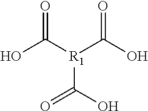 | III |
| 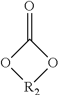 | IV |

R, R₁, and R₂, are, independently, selected from the group consisting of alkylenes, unsaturated alkylenes, bis alkylene ethers, cycloalkylenes, and arylenes. The terms “alkylene” and “arylene” as used herein are intended to include substituted and non-substituted groups such as hydrocarbons (e.g., alkyl groups) as well as substituents such as halogens, halocarbons, nitro and ether or oxygen groups (e.g., oxyalkylene). R, R₁, and R₂ can contain between 1 and 50 carbons.

Examples of linkers that are useful herein include malonic acid, succinic acid, gluatric acid, adipic acid, pimelic acid, oleic acid dimer, sebacic acid, suberic acid, azelaic acid, fumaric acid, citric acid, their corresponding anhydrides, and mixtures thereof.

In one aspect, the polyfunctional carboxylic acid has formula II and R contains from 2 to 14 carbon atoms, or contains 2, 3, 4, 5, 6, 7, 8, 9, 10, 11, 12, 13, Or 14 carbon atoms. In a further aspect, the polyfunctional carboxylic acid is succinic acid, which has formula II where R is an unsubstituted alkyl chain with 2 methylene groups (—CH₂CH₂—). In another aspect, the anhydride is succinic anhydride, which has formula IV where R₂ is an unsubstituted alkyl chain with 2 methylene groups (—CH₂CH₂—).

In one aspect, component (b) is D-alpha tocopheryl polyethylene glycol succinate, wherein the polyethylene glycol of D-alpha tocopheryl polyethylene glycol succinate is from 200 Da to 2,000 Da, 800 Da to 1200 Da, or about 1,000 Da.

### Poloxamer

One or more poloxamers (component (c)) are present in the compositions described herein. As used herein, a “poloxamer” is a nonionic triblock copolymer. The central block in a poloxamer is hydrophobic and comprises polypropylene oxide, while the outer two blocks are hydrophilic and consist of polyethylene oxide chains. Poloxamers are known to self-assemble in a temperature-dependent manner and may, for example, form gels at higher temperatures while remaining liquid at lower temperatures. In some aspects, poloxamers can increase the water solubility of hydrophobic substances.

In one aspect, the poloxamer has the formula

HO(C₂H₄O)_{b}(C₃H₆O)ₐ(C₂H₄O)_{b}H

wherein a is from 5 to 100, 5 to 50, 25 to 50, or from 25 to 35; b is from 5 to 100, 20 to 100, 50 to 100, 50 to 80, or 70 to 80. In a further aspect, a is from 25 to 35 and b is from 70 to 80. In another aspect, the poloxamer has a molecular weight from 2,000 to 15,000, 3,000 to 14,000, or 4,000 to 12,000 Daltons. In another aspect, a is about 29 and b is about 75 and the poloxamer has a molecular weight of about 8,400 Daltons.

In another aspect, the poloxamer has an oxyethylene content of about 70% to about 90%, from 75% to 85%, or from 79.9% to 83.7%.

Poloxamers useful herein are sold under the trade name PLURONIC® manufactured by BASF. In one aspect, PLURONIC® P188 or PLURONIC® P407 can be used as the poloxamer in the compositions disclosed herein. In a further aspect, the poloxamer has a polyoxypropylene molecular mass of 1,800 g/mol and 80% polyoxyethylene content. In an alternative aspect, the poloxamer has a polyoxypropylene molecular mass of 4,000 g/mol and 70% polyoxyethylene content.

In one aspect, the poloxamer assists with nanodroplet formation. In another aspect, the molecular weight and composition of the poloxamer contributes to the generation of nanodroplets of the desired size. In an alternative aspect, the poloxamer coats the surfaces of nanodroplets to avoid detection and clearance of the nanodroplets by the reticuloendothelial system.

### Polyalkylene Glycol

In one aspect, the nanodroplets described herein contain a polyalkylene glycol (component (d)). In one aspect, the polyalkylene glycol can be a homopolymer of ethylene oxide, a homopolymer of propylene oxide, a block copolymer or reverse block copolymer of ethylene oxide and propylene oxide, or a random copolymer of ethylene oxide and propylene oxide.

In one aspect, the polyalkylene glycol has a molecular weight of from 100 Da to 2,000 Da, or is 100, 200, 300, 400, 500, 600, 700, 800, 900, 1,000, 1,500, or 2,000 Da, where any number can be the upper and/or lower endpoint of a range (e.g., from 200 to 600 Da or from 300 to 500 Da). In one aspect, the polyalkylene glycol is a homopolymer of polyethylene glycol and has a molecular weight of 400 Da (e.g., is PEG400).

In one aspect, the polyalkylene glycol assists with nanodroplet formation. In another aspect, the molecular weight of the polyalkylene glycol contributes to the generation of nanodroplets of the desired size. In an alternative aspect, the polyalkylene glycol coats the surfaces of nanodroplets to avoid detection and clearance of the nanodroplets by the reticuloendothelial system.

### Lyoprotectant

In certain aspects, the nanodroplet compositions are lyophilized or freeze-dried to be reconstituted later. In these aspects, the nanodroplet compositions include a lyoprotectant. As used herein, a “lyoprotectant” is a molecule that protects material that has been freeze-dried or lyophilized. Examples of lyoprotectants include, but are not limited to, sugars, sugar alcohols, or other polyhydroxy compounds. Lyoprotectants useful herein can be natural or synthetic products. In a further aspect, the lyoprotectant also acts as an osmoregulator.

In one aspect, the lyoprotectant is a sugar or sugar alcohol. In a further aspect, the sugar or sugar alcohol can be mannitol, sucrose, glucose, or a combination thereof. In one aspect, the lyoprotectant is mannitol.

### Anti-Cancer Agent

The nanodroplets described herein may include one or more anti-cancer agents incorporated within the nanodroplets. As used herein, an “anti-cancer” agent is a compound or composition used in chemotherapy to kill cancer cells in the body of a subject, to slow the growth of cancer in a subject, to keep a cancer from spreading in a subject, or to prevent the return of a tumor that has been surgically removed. Anti-cancer agents may operate by a variety of methods including, but not limited to, by alkylating DNA (which can interfere with coiling and recognition by DNA replication enzymes), by interfering with the production of DNA, by interfering with the production of proteins in cancer cells, by preventing cancer cells from dividing, or by slowing the growth of a cancer that depends on hormones.

Examples of anti-cancer agents include, but are not limited to, platinum compounds (e.g., cisplatin, carboplatin, oxaliplatin), alkylating agents (e.g., cyclophosphamide, ifosfamide, chlorambucil, nitrogen mustard, thiotepa, melphalan, busulfan, procarbazine, streptozocin, temozolomide, dacarbacine, bendamustine), antitumor antibiotics (e.g., daunorubicin, doxorubicin, idarubicin, epirubicin, mitoxantrone, bleomycin, mitomycin C, plicamycin, dactinomycin, amphotericin B, nystatin), taxanes (e.g., paclitaxel and docetaxel), antimetabolites (e.g., 5-fluorouracil, cytarabine, premetrexed, thioguanine, floxuridine, capecitabine, gemcitabine, and methotrexate), nucleoside analogues (e.g., fludarabine, clofarabine, cladribine, penostatin, and nelarabine), topoisomerase inhibitors (e.g., topotecan and irinotecan), hypomethylating agents (e.g., azacitidine and decitabine), proteosome inhibitors (e.g., bortezomib), epipodophyllotoxins (e.g., etoposide and teniposide), DNA synthesis inhibitors (e.g., hydroxyurea), vinca alkaloids (e.g., vincristine, vindesine, vinorelbine, and vinblastine), tyrosine kinase inhibitors (e.g., imatinib, dasatinib, nilotinib, sorafenib, sunitinib), monoclonal antibodies (e.g., rituximab, cetuximab, panetumumab, tositumomab, trastuzumab, alemtuzumab, gemtuzumab, ozogamicin, bevacizumab), nitrosoureas (e.g., carmustine, fotemustine, and lumustine), enzymes (e.g., L-asparaginase), biological agents (e.g., interferons and interleukins), hexamethylmelamine, mitotane, angiogenesis inhibitors (e.g., thalidomide, lenalidomide), steroids (e.g., prednisone, dexamethasone, betulinic acid, testosterone, estrogen, progesterone, and prednisolone), hormonal agents (e.g., tamoxifen, faloxifene, leuprolide, bicalutamide, granisetron, flutamide), aromatase inhibitors (e.g., letrozole and anastrozole), arsenic trioxide, tretinoin, nonselective cyclooxygenase inhibitors (e.g., nonsteroidal anti-inflammatory agents, salicylates, aspirin, piroxicam, ibuprofen, indomethacin, naprosyn, diclofenac, tolmetin, ketoprofen, nabumetone, oxaprozin), selective cyclooxygenase-2 (COX-2) inhibitors, diazepam, propofol, 2,3-mercaptopropanol, or any combination thereof. In one aspect, the anti-cancer agent is docetaxel. In another aspect, a tocopherol or tocotrienol that is covalently bonded to a polyalkylene glycol is the anti-cancer agent.

In one aspect, when an anti-cancer agent is incorporated into the nanodroplets described herein, the selection of the anti-cancer agent can vary depending upon the solvents used to produce the nanodroplets. In one aspect, an anti-cancer agent can be incorporated into the nanodroplets when the anti-cancer agent and components use to produce the nanodroplets are soluble in the same organic solvent.

### Preparation of the Nanodroplet Compositions

In one aspect, the nanodroplet compositions can be prepared by dissolving the tocotrienol, the tocopherol or tocotrienol covalently bonded to a polyalkylene glycol, the poloxamer, and the polyalkylene glycol in an organic solvent. In one aspect, the solvent is methanol, ethanol, chloroform, dichloromethane, or a combination thereof. In another aspect, the solvent has a boiling point of less than 100° C. In a further aspect, the solvent is ethanol. As discussed above, when an anti-cancer agent is to be included in the nanodroplets, the anti-cancer agent is also dissolved in the organic solvent along with these other components. For example, docetaxel can be incorporated into the nanodroplets described herein due to its solubility in organic solvents.

In a further aspect, the solvent is then removed under vacuum with heating. In this aspect, the solvent-free composition forms a thin film. In a still further aspect, the thin film can be hydrolyzed with an aqueous solution of a lyoprotectant such as, for example, mannitol. In yet another aspect, the hydrolysis is carried out at room temperature until no viscous aggregate is observed. Further in this aspect, the lyoprotectant solution is 5% w/v. In a still further aspect, a volume of lyoprotectant solution is added such that the final concentration of tocotrienol in the nanodroplet composition is at or about 5 mg/mL.

In one aspect, following hydrolysis of the thin film, the nanodroplet composition is filtered with a 200 nm filter.

In one aspect, the nanodroplets as described herein have a Z-average diameter of from 10 to 100 nm as measured by dynamic light scattering (Zetasizer from Malvern Instruments; Malvern, UK). In another aspect, the nanodroplets with delta-tocotrienol have a Z-average diameter of about 10, 20, 30, 40, 50, 60, 70, 80, 90, or 100 nm as measured by dynamic light scattering, where any value can be a lower and/or upper endpoint of a range (e.g., 20 to 40 nm, 30 to 40 nm, etc.). In another aspect, the nanodroplets have a Z-average diameter of from 30 to 40 nm or of about 33 nm.

In one aspect, the dry weight ratio of component (b) to the tocotrienol of component (a) is from 5:1 to 20:1 or is 5:1, 6:1, 7:1, 8:1, 9:1, 10:1, 11:1, 12:1, 13:1, 14:1, 15:1, 16:1, 17:1, 18:1, 19:1, or 20:1. In another aspect, the dry weight ratio of the poloxamer of component (c) to the tocotrienol of component (a) is from 0.5:1 to 20:1, or is 0.5:1, 1:1, 1.5:1, 2:1, 2.5:1, 5:1, 7.5:1, 10:1, 15:1, or 20:1. In still another aspect, the dry weight ratio of the polyalkylene glycol of component (d) to the tocotrienol of component (a) is from 5:1 to 20:1 or is 5:1, 6:1, 7:1, 8:1, 9:1, 10:1, 11:1, 12:1, 13:1, 14:1, 15:1, 16:1, 17:1, 18:1, 19:1, or 20:1.

In a further aspect, the concentration of mannitol in the nanodroplet composition is at or about 5% (v/v) with respect to the aqueous solution. In one aspect, if the tocotrienol of component (a) is 5 mg/mL in the aqueous solution, the dry weight ratio of lyoprotectant to tocotrienol is from 2 to 10.

In another aspect, the nanodroplets also contain an anti-cancer agent. Further in this aspect, the dry weight of the anti-cancer agent to the tocotrienol of component (a) is from 0.1:1 to 2:1, or is 0.1:1, 0.2:1, 0.3:1, 0.4:1, 0.5:1, 0.6:1, 0.7:1, 0.8:1, 0.9:1, 1:1, 1.1:1, 1.2:1, 1.3:1, 1.4:1, 1.5:1, 1.6:1, 1.7:1, 1.8:1, 1.9:1, or is 2:1.

In another aspect, provided herein are nanodroplets in an aqueous solution where the nanodroplets include:
(a) D-delta-tocotrienol or D-gamma-tocotrienol;
(b) D-alpha tocopheryl polyethylene glycol succinate, wherein the molecular weight of the polyethylene glycol is from 900 Da to 1,100 Da;
(c) a poloxamer with the formula

HO(C₂H₄O)_{b}(C₃H₆O)ₐ(C₂H₄O)_{b}H
wherein a is from 25 to 35 and b is from 70 to 80;
(d) polyethylene glycol having a molecular weight of from 350 Da to 450 Da; and
(e) a lyoprotectant, wherein the lyoprotectant is mannitol.

Further in this aspect, the dry weight ratio of D-alpha tocopheryl polyethylene glycol succinate to D-delta-tocotrienol or D-gamma-tocotrienol is from 10:1 to 15:1, or is 10:1, 11:1, 12:1, 13:1, 14:1, or 15:1.

Still further in this aspect, the dry weight ratio of poloxamer to D-delta-tocotrienol or D-gamma-tocotrienol is from 1:1 to 1.5:1, or is 1:1, 1.1:1, 1.2:1, 1.3:1, 1.4:1, or 1.5:1. Even further in this aspect, the dry weight ratio of polyethylene glycol to D-delta-tocotrienol or D-gamma-tocotrienol is from 10:1 to 15:1, or is 10:1, 11:1, 12:1, 13:1, 14:1, or 15:1.

Further in the above aspects, the molecular weight of polyethylene glycol in D-alpha tocopheryl polyethylene glycol succinate is at or about 1,000 Da. Still further in the above aspects, the molecular weight of polyethylene glycol in component (d) is about 400 Da.

In any of the above aspects, the nanodroplets have a Z-average diameter of from 20 nm to 50 nm, or have a Z-average diameter of 20 nm, 25 nm, 30 nm, 35 nm, 40 nm, 45 nm, or 50 nm. In one aspect the Z-average diameter of the nanodroplets is about 33 nm.

In some aspects, the nanodroplets further contain docetaxel at a dry weight ratio of docetaxel to tocotrienol (component a) is from 0.2:1 to 1:1, or is 0.2:1, 0.3:1, 0.4:1, 0.5:1, 0.6:1, 0.7:1, 0.8:1, 0.9:1, or 1:1.

### Pharmaceutical Compositions

Any of the nanodroplet compositions described herein can be combined with at least one pharmaceutically-acceptable carrier to produce a pharmaceutical composition suitable for administration to a subject. The pharmaceutical compositions can be prepared using techniques known in the art. In one aspect, the pharmaceutical composition is prepared by admixing the nanodroplets with a pharmaceutically-acceptable carrier.

Pharmaceutically-acceptable carriers are known to those skilled in the art. These most typically would be standard carriers for administration to humans, including solutions such as sterile water, saline, and buffered solutions at physiological pH.

Molecules intended for pharmaceutical delivery may be formulated in pharmaceutical compositions. Pharmaceutical compositions can include carriers, thickeners, diluents, buffers, preservatives, surface active agents, and the like, in addition to the molecule or nanodroplet composition of choice. Pharmaceutical compositions can also include one or more additional active ingredients such as antimicrobial agents, anti-inflammatory agents, anesthetics, and the like.

In some aspects, the nanodroplet compositions described herein are provided as a dry (lyophilized) powder and can be reconstituted in water or another appropriate vehicle for intravenous administration as described below.

The compositions described herein can be formulated in any excipient the patient or entity can tolerate to produce pharmaceutical compositions. Examples of such excipients include, but are not limited to, water, aqueous hyaluronic acid, saline, Ringer's solution, dextrose solution, Hank's solution, and other aqueous physiologically balanced salt solutions. Nonaqueous vehicles, such as fixed oils, vegetable oils such as olive oil and sesame oil, triglycerides, propylene glycol, polyethylene glycol, and injectable organic esters such as ethyl oleate can also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer, and Tris buffer, while examples of preservatives include thimerosal, cresols, formalin, and benzyl alcohol. In certain aspects, the pH can be modified depending upon the mode of administration. Additionally, the compositions can include carriers, thickeners, diluents, preservatives, surface active agents (surfactants), and the like, in addition to the compounds described herein.

### Cancer Treatment

The nanodroplets described herein are applicable for treating a variety of different types of cancers. In one aspect, the cancer includes prostate cancer, leukemia (e.g., acute myologenous leukemia, acute promyelocytic leukemia, acute lymphoblastic leukemia, chronic myelogenous leukemia, chronic lymphocytic leukemia, hairy cell leukemia, plasma cell leukemia), myeloproliferative disorders (e.g., essential thrombocytosis, polythemia vera, primary myelofibrosis), myelodysplastic syndromes, lymphoma (Hodgkin and non-Hodgkin), testicular cancer, head and neck cancer, esophageal cancer, stomach cancer, liver cancer, cancer of the small intestine, gall bladder cancer, rectal or anal cancer, sarcomas, uterine or cervical cancer, bladder cancer, bone cancer, renal cancer, melanoma and other skin cancers, colon cancer, ovarian cancer, lung cancer, cancers of the central nervous system, multiple myeloma, or breast cancers.

The nanodroplets can be administered to the subject intravenously, subcutaneously, or intratumorally. In one aspect, a cancerous tumor in a subject will be reduced in size upon administration of the nanodroplet compositions. In another aspect, a cancerous tumor in a subject will be eliminated upon administration of the nanodroplet compositions.

In one aspect, the nanodroplets described herein can be administered to a subject in the absence of an anti-cancer agent in order to treat cancer in the subject. In other words, the nanodroplets alone reduce the rate if tumor growth. Moreover, the nanodroplets when adminstered alone have no negative side-effects. Indeed, as demonstrated in the Examples, the nanodroplets as described herein do not result in significant weight loss over time when adminstered to the subject.

In a further aspect, the dosage of tocotrienol administered to the subject is from 20 mg/kg to 100 mg/kg per single administration, or is 20 mg/kg, 30 mg/kg, 40 mg/kg, 50 mg/kg, 60 mg/kg, 70 mg/kg, 80 mg/kg, 90 mg/kg, or 100 mg/kg per single administration. In another aspect, the dosage of anti-cancer agent administered to the subject is from 5 mg/kg to 30 mg/kg per single administration, or is 5 mg/kg, 10 mg/kg, 15 mg/kg, 20 mg/kg, 25 mg/kg, or 30 mg/kg per single administration.

In another aspect, the nanodroplets described herein can be co-administered with an anti-cancer agent, where the anti-cancer agent is not incorporated within the nanodroplets (i.e., the anti-cancer agent is administered separately from the nanodroplets). In one aspect, the anti-cancer agent can be administered at the same time the nanodroplets are administered to the subject. In another aspect, the nanodroplets are administered before and/or after administration of the anti-cancer agent.

In one aspect, the nanodroplets can be adminstered within 1 to 3 days prior to the administration of the anti-cancer agent. In another aspect, the nanodroplets can be adminstered within 1 to 3 days after the administration of the anti-cancer agent. In a further aspect, the nanodroplets can be adminstered within 1 to 3 days prior to and after the administration of the anti-cancer agent.

In another aspect, the nanodroplets when used alone or in combination with an anti-cancer agent can be administered to the subject from 1 to 3 times per week. In one aspect, the nanodroplets are administered to the subject at least 2 times per week when administered with or without an anti-cancer agent. In another aspect, the administration schedule in Table 3 can be used herein.

TABLE 3
Initial Administration of Anti-Cancer Agent

| | Second | Second | Third |
|---|---|---|---|
| Administration | Administration | Administration | Administration |
| of Nanodroplets | of Anti-Cancer | of Nanodroplets | of Anti-Cancer |
| after Initial | Agent after | after Second | Agent after |
| Administration | Initial | Administration | Second |
| of Anti-Cancer | Administration | of Anti-Cancer | Administration |
| Agent | of Nanodroplets | Agent | of Nanodroplets |
| 3 to 7 Days | 1 to 3 Days | — | — |
| 3 to 7 Days | 1 to 3 Days | 4 to 8 Days | 1 to 3 Days |

The nanodroplets described herein possess numerous properties when it comes to the treatment of cancer. As discussed above, the nanodroplets in the absence of an anti-cancer agent can be used to treat cancer without the risk of adverse side effects (e.g., weight loss).

Moreover, when used in combination with an anti-cancer agent, the nanodroplets described herein can reduce or prevent undesirable side effects associated with the anti-cancer agent. Examples of such side effects include, but are not limited to, fatigue; pain including headaches, muscle pain, stomach pain, or pain from nerve damage; mouth and/or throat sores; gastrointestinal effects including loss of appetite, diarrhea, nausea, vomiting, or constipation; blood disorders including anemia, leukopenia, or thrombocytopenia; nervous system effects including tingling, burning, weakness or numbness, loss of balance, or tremors; cognitive dysfunction; sexual or reproductive dysfunction; hair loss; weight loss; damage to other organ systems including the kidneys, liver, and related glands and organs; and combinations thereof.

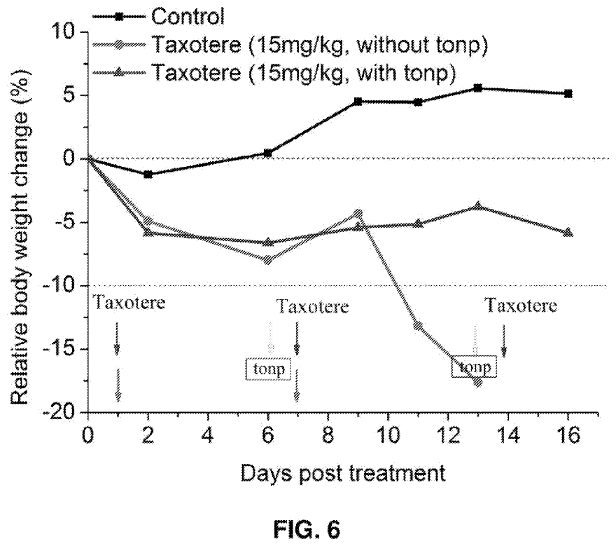

One problem associated with chemotherapy is that the subject can lose a significant amount of weight. The nanodroplets described herein can prevent this. For example, the anti-cancer agent docetaxel (TAXOTERE®) can cause significant weight loss. As demonstrated in the Examples, when mice are co-administered nanodroplets of the present invention and docetaxel, the mice did not exhibit weight loss over time compared to mice just administered docetaxel (FIG. 6).

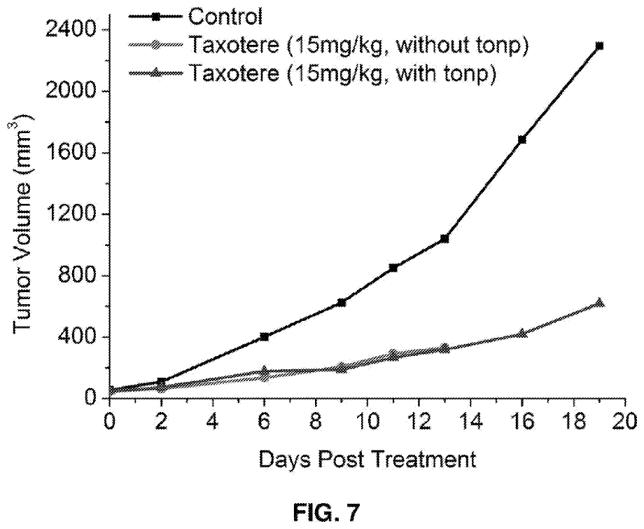

Thus, the ability of the nanodroplets to reduce the side effects of anti-cancer agents enhances the ability of the anti-cancer agent to treat cancer in a subject over extended periods of time. An example of this is shown in FIG. 7, where after two injections of TAXOTERE®, the mice without nanodroplets (Tonp) suffered a severe body weight drop and the treatment ended on the 13^{th} day with 71% tumor growth inhibition (TGI). For the group treated with TAXOTERE® and Tonp, on the 19^{th} day of the study, the tumor growth inhibition of the combined treatment was 76%, showing the synergistic effect of the nanodroplets with TAXOTERE®. Tumor growth was inhibited approximately equally in both TAXOTERE® groups, showing that the nanodroplets do not decrease the efficacy of TAXOTERE®. Experimental results are provided in the Examples below.

### EXAMPLES

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. Numerous variations and combinations of reaction conditions (e.g., component concentrations, desired solvents, solvent mixtures, temperatures, pressures, and other reaction ranges and conditions) can be used to optimize the product purity and yield obtained from the described process. Only reasonable and routine experimentation will be required to optimize such process conditions.

### Example 1: Preparation of the Nanodroplets

### Components

A sample nanodroplet composition was produced using delta-tocotrienol (d-T3), d-alpha tocopheryl polyethyleneglycol 1000 succinate (TPGS), PLURONIC® P188, PEG400, mannitol, and water.

### Nanodroplet Formation

The nanodroplets were generated according to the following procedure:
1. d-T3, TPGS, PLURONIC® P188, and PEG400 were dissolved in an organic solvent.
2. The solvent was removed under vacuum with heating; this resulted in the formation of a thin film.
3. The thin film was hydrolyzed using an aqueous solution of mannitol at room temperature.
4. The resultant solution was filtered with a 200 nm filter.

### Organic Solvent

Various organic solvents were evaluated, including methanol, ethanol, chloroform, and dichloromethane, with ethanol being preferred. The ideal organic solvent must be able to dissolve d-T3, TPGS, and PLURONIC® P188 and should have a boiling point of less than 100° C. The solvent is preferably removed while heating to 50° C. under vacuum.

### Poloxamer

Various poloxamers were evaluated for their abilities to aid in nanodroplet formation and/or uptake of anti-cancer agent into the nanodroplets. Among these were PLURONIC® P188 and PLURONIC® P407, with PLURONIC® P188 exhibiting more desirable results.

### Lyoprotectant

Various lyoprotectants were evaluated, with mannitol being preferred. An aqueous solution with a 5% (w/v) concentration of mannitol was determined to be most effective as lyoprotectant as well as thin film hydrolysis. Thin film hydrolysis with an aqueous solution of mannitol was carried out at room temperature until no viscous aggregate is visible.

The volume of aqueous solution of mannitol can be used to control the concentration of d-T3 in the final solution, with the preferred concentration of d-T3 being 5 mg/mL.

### Anti-Cancer Agent

Some nanodroplets were prepared using an anti-cancer agent as an additional component. When used, the anti-cancer agent was dissolved in the same organic solvent as d-T3, TPGS, and the poloxamer. In some experiments, docetaxel was the anti-cancer agent used.

### Preferred Compositions

Table 4 below presents preferred identities and ratios of the components discussed above for nanodroplet compositions.

TABLE 4
Preferred Nanodroplet Compositions

| | | Amount per mL of | |
|---|---|---|---|
| Component | Weight Ratio with Respect to d-T3 | Nanodroplet Solution | |
| d-T3 | — | 2-5 | mg |
| TPGS | 5:1 to 20:1, preferred 10:1 to 15:1 | 30-75 | mg |
| PLURONIC ® P188 | 0.5:1 to 2:1, preferred 1:1 to 1.5:1 | 2-5 | mg |
| PEG400 | 5:1 to 20:1, preferred 10:1 to 15:1 | 20-50 | mg |

| | | |
|---|---|---|
| Docetaxel (if used) | 0.1:1 to 2:1, preferred 0.2:1 to 1:1 | — |
| 5% (w/v) mannitol | sufficient for d-T3 concentration of 5 | 5 mg/mL mannitol |
| aqueous solution | mg/mL | |
| Water for injection | — | q.s. |

### Example 2: Characterization of Nanodroplets

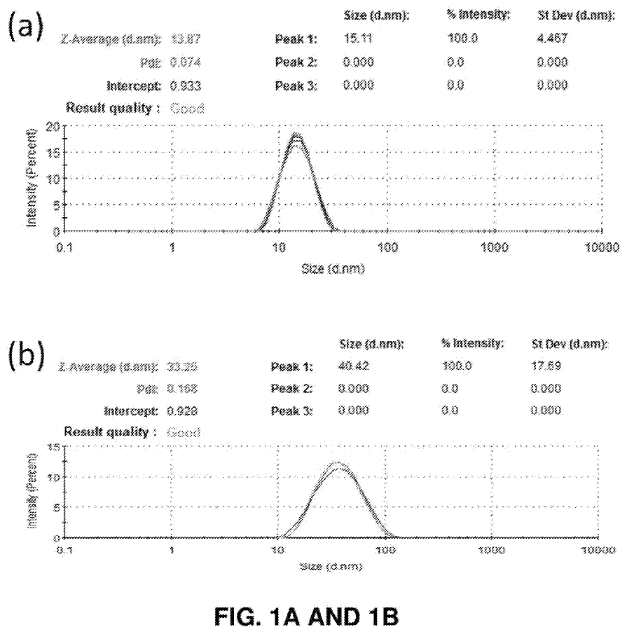

Nanodroplets were characterized by dynamic light scattering (Zetasizer from Malvern Instruments, Malvern, UK). Nanodroplets typically had Z-average diameters of 10-20 nm, or about 14 nm, without d-T3. With d-T3, nanodroplets typically had Z-average diameters of 25-50 nm, or about 33 nm (FIG. 1).

### Example 3: Toxicity of Nanodroplets

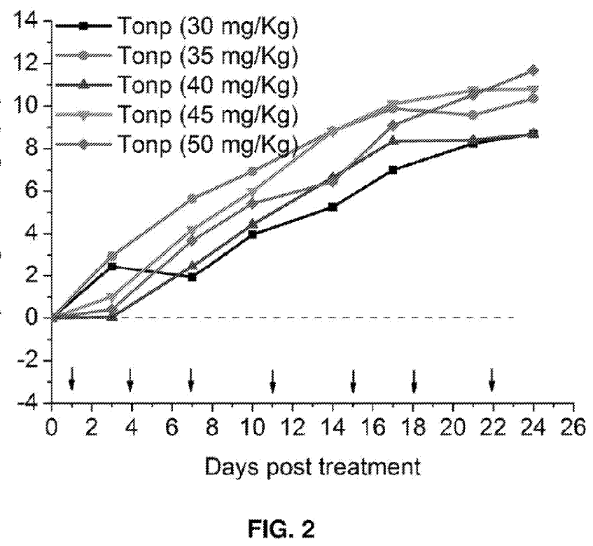

Toxicity of the nanodroplet formulations was assessed with respect to groups of mice without tumors as follows. Nanodroplet compositions as described above were prepared. Groups of mice (n=5) were injected through the tail vein twice per week with different dosages of d-T3 per injection. At the end of the trial, all groups of mice increased in body weight, indicating low toxicity of the nanodroplet formulations (FIG. 2).

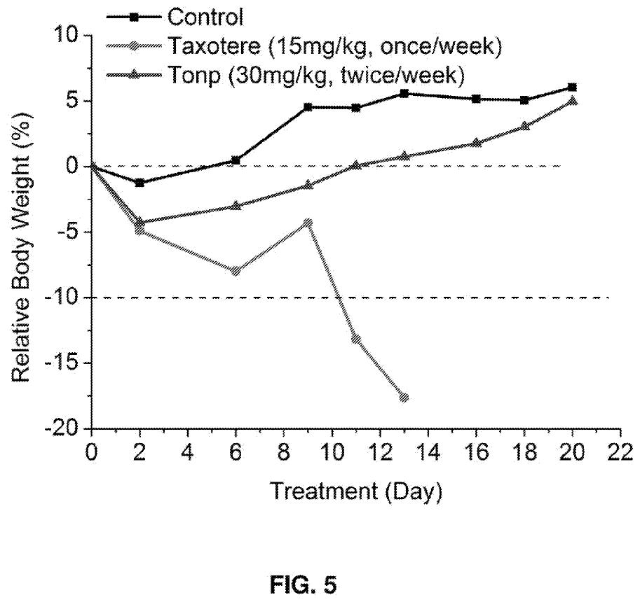

Toxicity of the nanodroplet formulations was further assessed with respect to groups of mice with tumors. NCI-H460 human lung cancer cells were injected subcutaneously on the backs of nude mice to establish a tumor model. When tumor volume reached approximately 50 mm³, mice were divided into treatment groups, which typically consisted of five mice. Mice were injected through the tail vein twice per week for three weeks with 30 mg/kg of Tonp or once per week with a dose of 15 mg/kg of TAXOTERE®. After about two weeks of treatment, mice in the TAXOTERE® group had significantly decreased in body weight while Tonp-treated mice did not have significantly different body weights from untreated controls (FIG. 5).

### Example 4: Efficacy of Nanodroplets

NCI-H460 human lung cancer cells were injected subcutaneously on the backs of nude mice to establish a tumor model. When tumor volume reached approximately 50 mm³, mice were divided into treatment groups, which typically consisted of five mice.

### Tumor Growth Inhibition

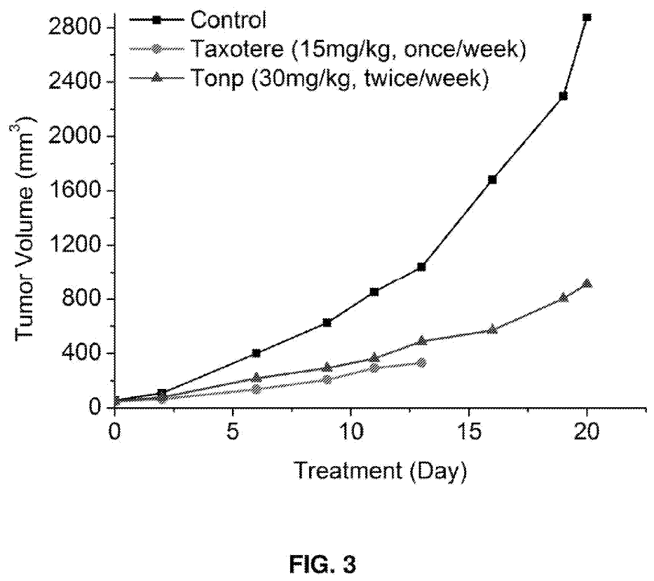

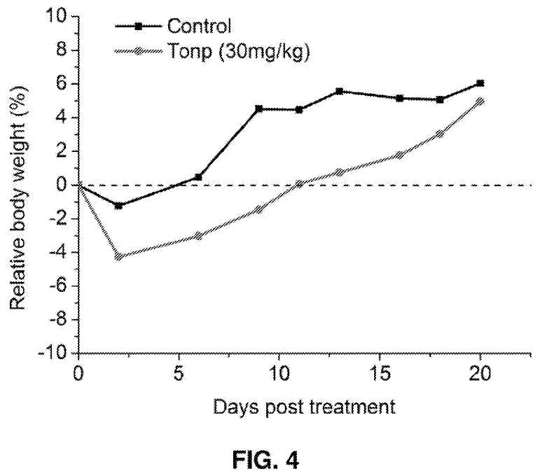

In one experiment, tumor-bearing mice were either left untreated, were treated with TAXOTERE®, or were treated with Tonp nanodroplets. Mice were injected through the trail vein once per week with 15 mg/kg of TAXOTERE® or twice per week for three weeks with a dose of 30 mg/kg of Tonp. On the 20^{th} day, tumor growth inhibition of Tonp treatment was 70%, meaning d-T3 is at least as effective as TAXOTERE® over the course of the study period, with both treatments being more effective than no treatment as in the control group (FIG. 3). Further, body weight for tumor-bearing mice injected twice per week for three weeks with 30 mg/kg of Tonp did not have significantly different body weights as compared to control mice (FIG. 4).

### Chemoprotective Effects

Therapy with Tonp nanodroplets in combination with TAXOTERE® treatment resulted in chemoprotective effects when compared to TAXOTERE® treatment alone or untreated controls. One group of tumor-bearing mice was injected with TAXOTERE® through the tail vein once per week at a dose of 15 mg/kg per animal. A second group of tumor-bearing mice was injected with TAXOTERE® and Tonp through the tail vein once per week at doses of 15 mg/kg and 20 mg/kg, respectively. Tonp, when used, was injected one day before TAXOTERE® starting just before the second injection of TAXOTERE®. After two injections of TAXOTERE®, the group without Tonp showed a severe body weight drop and the treatment ended on the 13^{th} day of the study. However, one injection of Tonp inhibited this drop of body weight and allowed for extended treatment. The severe weight loss caused by TAXOTERE® treatment was mediated by simultaneous treatment with d-T3 nanodroplets, thus demonstrating the chemoprotective effects of Tonp (FIG. 6).

### Tumor Growth Inhibition of Combined TAXOTERE® and Tonp Treatment

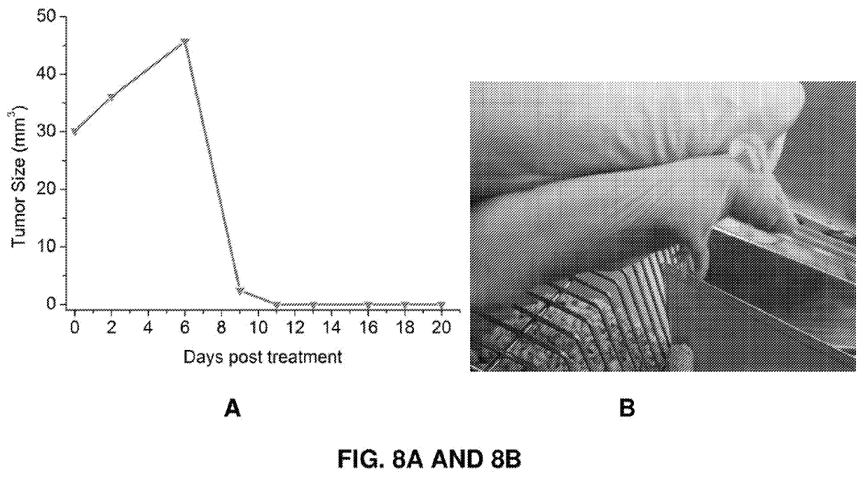

One group of tumor-bearing mice was injected with TAXOTERE® through the tail vein once per week at a dose of 15 mg/kg per animal. A second group of tumor-bearing mice was injected with TAXOTERE® and Tonp through the tail vein once per week at doses of 15 mg/kg and 20 mg/kg, respectively. Tonp, when used, was injected one day before TAXOTERE® starting just before the second injection of TAXOTERE®. After two injections of TAXOTERE®, the group without Tonp showed a severe body weight drop and the treatment ended on the 13^{th} day of the study with 71% tumor growth inhibition. For the group treated with both TAXOTERE® and Tonp, on the 19^{th} day of the study, tumor growth inhibition of the combined treatment was 76%, showing the synergistic effect of Tonp with TAXOTERE®. Tumor growth was inhibited approximately equally in both TAXOTERE® groups, showing that Tonp use does not decrease the efficacy of TAXOTERE® (FIG. 7). In one instance, a tumor-bearing mouse in the combined TAXOTERE® and Tonp treatment group showed complete disappearance of the tumor by the 11^{th} day of treatment; the tumor did not recur by the end of the trial (FIG. 8).

Throughout this publication, various publications are referenced. The disclosures of these publications in their entireties are hereby incorporated by reference into this application in order to more fully describe the methods, compositions, and compounds herein.

Various modifications and variations can be made to the materials, methods, and articles described herein. Other aspects of the materials, methods, and articles described herein will be apparent from consideration of the speculation and practice of the materials, methods, and articles disclosed herein. It is intended that the specification and examples be considered as exemplary.

1. Nanodroplets comprising
(a) a tocotrienol;
(b) a tocopherol or tocotrienol comprising a polyalkylene glycol covalently bonded to the tocopherol or tocotrienol;
(c) a poloxamer; and
(d) a polyalkylene glycol.

2. The nanodroplets of claim 1, wherein the tocotrienol is alpha-tocotrienol, beta-tocotrienol, gamma-tocotrienol, delta-tocotrienol, or any combination thereof.

3. The nanodroplets of claim 1, wherein component (a) is D-delta-tocotrienol.

4. The nanodroplets of claim 1, wherein component (a) is D-gamma-tocotrienol.

5. The nanodroplets of claim 1, wherein the tocopherol of component (b) is alpha-tocopherol, beta-tocopherol, gamma-tocopherol, delta-tocopherol, or any combination thereof.

6. The nanodroplets of claim 1, wherein the tocotrienol of component (b) is alpha-tocotrienol, beta-tocotrienol, gamma-tocotrienol, delta-tocotrienol, or any combination thereof.

7. The nanodroplets of claim 1, wherein component (b) is a tocopherol comprising polyethylene glycol covalently bonded to the tocopherol.

8. The nanodroplets of claim 7, wherein the polyethylene glycol has a molecular weight from 200 Da to 2,000 Da.

9. The nanodroplets of claim 1, wherein component (b) is D-alpha tocopheryl polyethylene glycol succinate, wherein the polyethylene glycol of D-alpha tocopheryl polyethylene glycol succinate is from 200 Da to 2,000 Da.

10. The nanodroplets of claim 1, wherein the poloxamer comprises a polyethylene oxide-polypropylene oxide-polyethylene oxide triblock copolymer.

11. The nanodroplets of claim 1, wherein the poloxamer has the formula
HO(C₂H₄O)_{b}(C₃H₆O)ₐ(C₂H₄O)_{b}H
wherein a is from 5 to 100 and b is from 5 to 100.

12. The nanodroplets of claim 11, wherein a is from 25 to 35 and b is from 70 to 80.

13. The nanodroplets of claim 1, wherein the polyalkylene glycol (component (d)) has a molecular weight from 100 Da to 2,000 Da.

14. The nanodroplets of claim 1, wherein the nanodroplets further comprise a lyoprotectant.

15. The nanodroplets of claim 14, wherein the lyoprotectant comprises a sugar.

16. The nanodroplets of claim 15, wherein the sugar comprises mannitol, sucrose, glucose, or any combination thereof.

17. The nanodroplets of claim 15, wherein the sugar is mannitol.

18. The nanodroplets of claim 1, wherein the nanodroplets further comprise an anti-cancer agent selected from the group consisting of paclitaxel, doxorubicin, gemcitabine, cisplatin, methotrexate, 5-fluorouracil, betulinic acid, amphotericin B, diazepam, nystatin, propofol, testosterone, estrogen, prednisolone, prednisone, 2,3 mercaptopropanol, progesterone, docetaxel, or any combination thereof.

19. The nanodroplets of claim 18, wherein the anti-cancer agent is docetaxel.

20. The nanodroplets of claim 1, wherein the dry weight ratio of component (b) to the tocotrienol (component a) is from 5:1 to 20:1.

21. The nanodroplets of claim 1, wherein the dry weight ratio of poloxamer to the tocotrienol (component a) is from 0.5:1 to 2:1.

22. The nanodroplets of claim 1, wherein the dry weight ratio of polyalkylene glycol (component d) to the tocotrienol (component a) is from 5:1 to 20:1.

23. The nanodroplets of claim 1, wherein the nanodroplets further comprise an anti-cancer agent, wherein the dry weight ratio of anti-cancer agent to tocotrienol (component a) is from to 0.1:1 to 2:1.

24. The nanodroplets of claim 1, wherein the nanodroplets comprise an aqueous composition comprising
(a) D-delta-tocotrienol or D-gamma-tocotrienol;
(b) D-alpha tocopheryl polyethylene glycol succinate, wherein the molecular weight of polyethylene glycol is from 900 Da to 1,100 Da;
(c) the poloxamer has the formula
HO(C₂H₄O)_{b}(C₃H₆O)ₐ(C₂H₄O)_{b}H
wherein a is from 25 to 35 and b is from 70 to 80;
(d) polyethylene glycol having a molecular weight from 350 Da to 450 Da; and
(e) a lyoprotectant, wherein the lyoprotectant is mannitol.

25. The nanodroplets of claim 24, wherein the dry weight ratio of D-alpha tocopheryl polyethylene glycol succinate to D-delta-tocotrienol or D-gamma-tocotrienol is from 10:1 to 15:1.

26. The nanodroplets of claim 25, wherein the dry weight ratio of poloxamer to D-delta-tocotrienol or D-gamma-tocotrienol is from 1:1 to 1.5:1.

27. The nanodroplets of claim 26, wherein the dry weight ratio of polyethylene glycol to D-delta-tocotrienol or D-gamma-tocotrienol is from 10:1 to 15:1.

28. The nanodroplets of claim 1, wherein the molecular weight of polyethylene glycol in D-alpha tocopheryl polyethylene glycol succinate is about 1,000 Da.

29. The nanodroplets of claim 1, wherein the molecular weight of polyethylene glycol (component d) is about 400 Da.

30. The nanodroplets of claim 1, wherein the nanodroplets have a Z-average diameter from 20 nm to 50 nm.

31. The nanodroplets of claim 1, wherein the nanodroplets further comprise docetaxel, wherein the dry weight ratio of docetaxel to component (a) is from to 0.2:1 to 1:1.

32. The nanodroplets of claim 1, wherein the nanodroplets comprise a dry powder.

33. A pharmaceutical composition comprising the nanodroplets of claim 1 and a pharmaceutically acceptable carrier.

34. A method for treating cancer in a subject comprising administering to the subject the nanodroplets of claim 1 and an anti-cancer agent.

35. The method of claim 34, wherein nanodroplets are administered to the subject before the administration of the anti-cancer agent.

36. The method of claim 34, wherein nanodroplets are administered to the subject after the administration of the anti-cancer agent.

37. The method of claim 34, wherein nanodroplets are administered to the subject before and after the administration of the anti-cancer agent.

38. A method for treating cancer in a subject comprising administering to the subject the nanodroplets of claim 1, wherein the nanodroplets further comprises an anti-cancer agent.

39. The method of claim 38, wherein the anti-cancer agent is docetaxel.

40. The method of claim 34, wherein the cancer is pancreatic cancer, lung cancer, breast cancer, ovarian cancer, prostate cancer, or colon cancer.

41. The method of claim 34, wherein the nanodroplets are administered to the subject by intravenously, subcutaneously, or intratumorally.

42. The method of claim 34, wherein the dosage of tocotrienol administered to the subject is from 20 mg/kg to 100 mg/kg per single administration.

43. The method of claim 34, wherein the dosage of the anti-cancer agent administered to the subject is from 5 mg/kg to 30 mg/kg per single administration.

44. The method of claim 34, wherein the nanodroplets are administered to the subject at least two times per week.

45. The method of claim 34, wherein the nanodroplets reduce one or more side-effects of the anti-cancer agent.

46. The method of claim 45, wherein the side-effect is weight loss.

47. A method for reducing a tumor in a subject comprising administering to the subject the nanodroplets of claim 1.
